(19) United States
(12) Patent Application Publication
(10) Pub. No.: US 2014/0284468 A1
(43) Pub. Date: Sep. 25, 2014

(54) Cancer patient selection for administration of therapeutic agents using mass spectral analysis of blood-based samples

(71) Applicant: Biodesix, Inc., Boulder, CO (US)

(72) Inventors: Julia Grigorieva, Steamboat Springs, CO (US); Heinrich Röder, Steamboat Springs, CO (US); Maxim Tsypin, Steamboat Springs, CO (US)

(21) Appl. No.: 14/295,783

(22) Filed: Jun. 4, 2014

Related U.S. Application Data
(63) Continuation of application No. 12/932,295, filed on Feb. 22, 2011.
(60) Provisional application No. 61/338,938, filed on Feb. 24, 2010.

(51) Int. Cl. G01N 33/68; H01J 49/40

(52) U.S. Cl. CPC G01N 33/6848; H01J 49/40
USPC 250/282

(57) ABSTRACT

Methods using mass spectral data analysis and a classification algorithm provide an ability to determine whether a solid epithelial tumor cancer patient is likely to benefit from a therapeutic agent or a combination of therapeutic agents targeting agonists of the receptors, receptors or proteins involved in MAPK (mitogen-activated protein kinase) pathways or the PKC (protein kinase C) pathway upstream from or at Akt or ERK/JNK/p38 or PKC, such as therapeutic agents targeting EGFR and/or HER2. The methods also provide the ability to determine whether the cancer patient is likely to benefit from the combination of a therapeutic agent targeting EFGR and a therapeutic agent targeting COX2; or whether the cancer patient is likely to benefit from the treatment with an NF-κB inhibitor.

### PRIORITY

This application is a continuation application of U.S. Ser. No. 12/932,295, filed on Feb. 22, 2011, which claims priority benefits under 35 U.S.C. §119(e) to U.S. Provisional patent application Ser. No. 61/338,938 filed Feb. 24, 2010, all of which are incorporated by reference herein.

### FIELD

This invention relates to methods and systems for predicting whether a cancer patient is likely or not likely to benefit from administration of certain types and classes of drugs, and/or combinations thereof. The methods and systems involve using mass spectral data obtained from a blood-based sample of the patient and a computer configured as a classifier operating on the mass spectral data.

### BACKGROUND

The assignee of the present invention, Biodesix, Inc, has developed a test known as VeriStrat which predicts whether Non-Small Cell Lung Cancer (NSCLC) patients are likely or not likely to benefit from treatment of Epidermal Growth Factor Receptor (EGFR) pathway targeting drugs. The test is described in U.S. Pat. No. 7,736,905, the content of which is incorporated by reference herein. The test is also described in Taguchi F. et al.¹, the content of which is also incorporated by reference herein. Additional applications of the test are also described in U.S. Pat. Nos. 7,858,390; 7,858,389 and 7,867,775, the contents of which are incorporated by reference herein.

In brief, the VeriStrat test is based on serum and/or plasma samples of cancer patients. Through a combination of MALDI-TOF mass spectrometry and data analysis algorithms implemented in a computer, it compares a set of eight integrated peak intensities at predefined m/z ranges with those from a training cohort, and generates a class label for the patient sample: either VeriStrat “good”, VeriStrat “poor”, or VeriStrat “indeterminate.” In multiple clinical validation studies it was shown that patients, whose pre-treatment serum/plasma was VeriStrat “good”, have significantly better outcome when treated with epidermal growth factor receptor inhibitor drugs than those patients whose sample results in a VeriStrat “poor” signature. In few cases (less than 2%) no determination can be made, resulting in a VeriStrat indeterminate label. VeriStrat is commercially available from Biodesix, Inc., the assignee of the present invention, and is used in treatment selection for non-small cell lung cancer patients.

Most modern biomarker-based tests are very specific with respect to tumor type and histology, specific interventions, and clinico-pathological factors. For example, genetic tests based on tumor tissue such as tests for mutations in the EGFR domain, KRAS mutations, and gene copy number analysis via Fluorescence In-Situ Hybridization (FISH) appear to work only in very specific indications. While EGFR mutations may give indications for gefitinib response in first line NSCLC cancer with adenocarcinoma, they do not exhibit similar utility for squamous cell carcinoma due to the extreme rarity of these mutations in this type of NSCLC. KRAS mutations can be associated with response to cetuximab in colorectal cancer, but attempts to transfer this to NSCLC have been unsuccessful. There are no known markers for EGFR-Inhibitor (EGFRI) benefit in squamous cell cancer of the head and neck (SCCHN). These limitations of genetic tests may be related to their focus on very specific mutations that are only a small part of the complex mechanism of carcinogenesis. Also, all of these tests are based on a reductionist point of view, i.e., reducing tumor biology to just tumor cells, and ignoring the important interplay between tumor cells and the tumor microenvironment consisting of endothelial cells of the vascular support system, extracellular matrix and the immune system components, such as inflammatory cells, and various chemokines and cytokines, involved in chronic inflammatory mechanisms associated with cancer.

### SUMMARY

In this document, we present our understanding, and evidence thereof, of which of the pathways in a tumor cell are involved in the distinct characteristics of VeriStrat “poor” epithelial tumors. The evidence for the understandings presented herein is based on several sources, including clinical evidence, phenomenological evidence, literature analysis, and molecular evidence based on mass spectrometry analysis of serum samples from cancer patients. The consequences of the realizations described herein can take the form of new methods (i.e., practical tests) for predicting whether cancer patients are likely or not likely to benefit from certain classes of drugs, or combinations thereof, described in detail below.

In brief, for patients identified as VeriStrat “poor”, the VeriStrat test measures the activation of one or more pathways downstream from the growth and survival factors receptors such as EGFR, likely candidate pathways include canonical and non-canonical MAPK (mitogen-activated protein kinase), Akt as well as reactions regulated by PKC (protein kinase C) (see FIG. 2). The variability with respect to outcomes of chemotherapy and placebo controls indicates that the activation of these pathways by themselves could lead to worse prognosis, and may point to the involvement of the NF-κB (nuclear factor kappa-light-chain-enhancer of activated B-cells)—an important transcription factor, regulating cellular responses and playing an essential role in inflammatory and immune responses, and in regulation of cell proliferation and survival. It is also known to be involved in the response to chemotherapy.

As a general matter, the VeriStrat test identifies a subset of population with worse prognosis and will predict differential benefit of solid epithelial tumor cancer patient from therapy with therapeutic agents or a combination of therapeutic agents targeting agonists of the receptors, receptors or proteins involved in MAPK pathways or the PKC upstream from or at Akt or ERK/JNK/p38 or PKC. EGFR inhibitors are the examples of such agents. Patients predicted to be likely to benefit from anti-EGFR agents are identified as VeriStrat “good” label; conversely patients predicted as not likely to benefit from anti-EGFR agents are identified with VeriStrat “poor” label. The term MAPK (mitogen-activated protein kinase) here is used as a name of at least three related cascades, not of a single enzyme (see FIG. 2).

As a corollary to the above statement, for patients that are associated with the VeriStrat “poor” label, VeriStrat test is diagnostic for “poor” patients as a subgroup of cancer patients with a poor prognosis. Indeed, the VeriStrat “poor” patients can be considered as having a different disease state from VeriStrat “good” patients.

Moreover, cancer patients having a VeriStrat “good” label are more likely to obtain more benefit from a therapy with therapeutic agent or a combination of therapeutic agents targeting agonists of the receptors, receptors or proteins involved in MAPK pathways; while patients having a VeriStrat “poor” label are not likely to obtain clinical benefit from therapy with such a therapeutic agent; on the other hand, VeriStrat “poor” patients are likely to exhibit benefit from a therapy or combination of therapies that prevents downstream, independent of the receptors, activation of these pathways.

Practical applications of this understanding can take several forms, as reflected in the appended claims. The methods involve obtaining mass spectral data of blood based samples from a cancer patient and analysis of the spectrum using a programmed computer functioning as a classifier. In one form, a method is disclosed of identifying a solid epithelial tumor cancer patient as being likely to benefit from treatment with a therapeutic agent or a combination of therapeutic agents targeting agonists of the receptors, receptors or proteins involved in MAPK pathways or the PKC (protein kinase C) pathway upstream from or at Akt or ERK/JNK/p38 or PKC or not likely to benefit from treatment with the therapeutic agent or the combination of therapeutic agents, comprising the steps of: a) obtaining a mass spectrum from a blood-based sample from the solid epithelial tumor cancer patient; b) performing one or more predefined pre-processing steps on the mass spectrum obtained in step a); c) obtaining integrated intensity values of selected features in said spectrum at one or more predefined m/z ranges after the pre-processing steps on the mass spectrum in step b) have been performed; and d) using the values obtained in step c) in classification algorithm using a training set comprising class-labeled spectra produced from blood-based samples from other solid tumor patients to identify the patient as being either likely or not likely to benefit from treatment with the therapeutic agent or a combination of therapeutic agents.

In another embodiment, a method is described for predicting whether a cancer patient is likely to benefit from administration of the combination of a COX2 inhibitor and a EGFR inhibitor, comprising the steps of:

a) obtaining a mass spectrum from a blood-based sample from the cancer patient;

b) performing one or more predefined pre-processing steps on the mass spectrum obtained in step a);

c) obtaining integrated intensity values of selected features in said spectrum at one or more predefined m/z ranges after the pre-processing steps on the mass spectrum in step b) have been performed; and

d) using the values obtained in step c) in classification algorithm using a training set comprising class-labeled spectra produced from blood-based samples from other solid epithelial tumor patients to identify the patient as being either likely or not likely to benefit from treatment by administration of a combination of a COX2 inhibitor and a EGFR inhibitor.

### BRIEF DESCRIPTION OF THE DRAWINGS

FIG. 1 is a flow chart showing the steps for performing the VeriStrat test on a blood-based sample of a patient.

FIG. 2 is a chart showing selected signal transduction pathways in human cells.

FIG. 3 is a representation of selected biological activity of serum amyloid A (SAA) isoforms and its possible role in cancer progression and therapy resistance.

FIG. 4 is a representation of EGFR signal transduction pathways, their interactions, and possible points of activation by SAA

FIG. 5 is a representation of ErbB family growth factor receptors, including EGFR, and their inhibitors, from Yarden Y, Shilo B Z. SnapShot: EGFR signaling pathway. Cell 2007; 131:1018

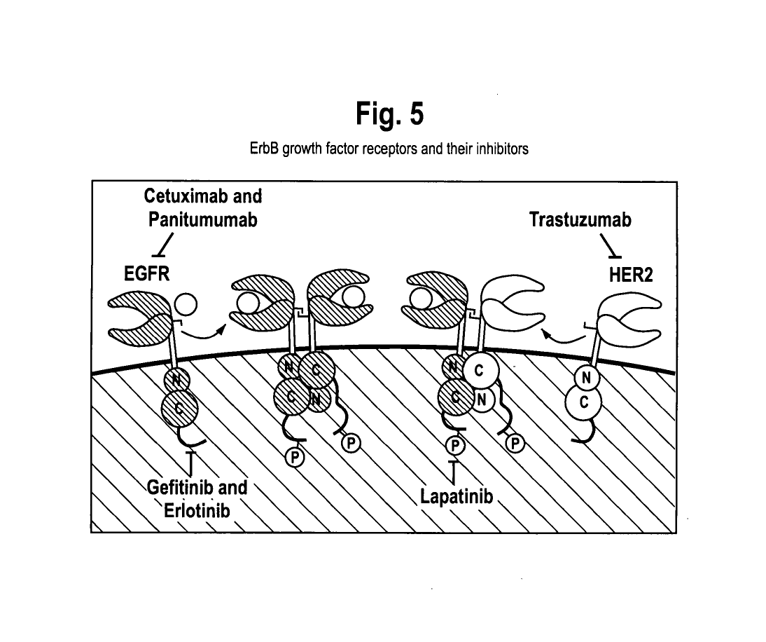

FIG. 6 is a forest plot showing the hazard ratios between VeriStrat Good and VeriStrat Poor patients by treatment arm for all published VeriStrat analyses.

FIG. 7 is a representation of Kaplan-Meier plots of overall survival (OS) of patents receiving different chemotherapy treatments and the VeriStrat labels (“good” and “poor”) for such patients.

FIG. 8 are plots of growth of gefitinib sensitive cell line HCC4006 and gefitinib resistant cell line A549 in VeriStrat “poor” and VeriStrat “good” serum in presence of different concentrations of gefitinib.

### DETAILED DESCRIPTION OF THE INVENTION

### Definitions

As used herein, the singular forms “a,” “an,” and “the” include plural referents unless the context clearly dictates otherwise.

As used herein, the term “solid epithelial tumor” includes but is not necessarily limited to NSCLC, SCCHN, breast cancer, renal cancer, pancreatic cancer, melanoma and colorectal cancer (CRC).

As used herein, the term “therapeutic agent or a combination of therapeutic agents targeting agonists of the receptors, receptors or proteins involved in MAPK pathways or the PKC upstream from or at Akt or ERK/JNK/p38 or PKC” includes but is not limited to therapeutic agent or agents targeting erbB receptors family, including EGFR (HER1), HER2, HER3, and HER4, VEGF Receptor (VEGFR2), Hepatocyte growth factor receptor (HGFR or MET), G-protein coupled receptors, Insulin-like Growth Factor (IGF) receptors, VEGF, Growth Factors such as TGFα and EGF, and any other protein upstream from or at Akt, or ERK/JNK/p38 MAPK or the PKC pathways. In addition, as used herein, the term “therapeutic agent or a combination of therapeutic agents targeting proteins at MAPK pathways or the PKC pathway upstream from or at Akt or ERK/JNK/p38 or PKC” includes known therapeutic agents, as well as therapeutic agents targeting these proteins that are yet to be discovered or disclosed. Moreover, the combination of therapeutic agents includes any combination of therapeutic agents, whether they have already been used in combination for treatment of solid epithelial tumors or not. It should be noted that even where an agent is identified as an inhibitor of a particular protein or pathway, such a classification is not meant to represent a description of its mechanism of action because the mechanism of action of many of these agents is not completely understood. As an example, but not as meant as an exhaustive list, these therapeutic agents include:

(1) TKIs (Tyrosine Kinase Inhibitors): There are many drugs currently on the market and in phase I-III clinical trials that are classified as small molecule Tyrosine Kinase Inhibitors (TKIs). TKIs may target specific molecular receptors, such as the Epidermal Growth Factor receptor (EGFR), and may also target multiple receptors (called “multiple kinase inhibitors”). These include but are not limited to erlotinib, gefitinib, sorafenib, sunitinib, pazopanib, imatinib, nilotinib, lapatinib.

Antibody-based inhibitors include Cetuximab (anti-EGFR), Panitumumab (anti-EGFR), Trastuzumab (anti-Her2).

(2) HGFR or MET inhibitors: Currently there is a long list of drugs in phase I-II trials, that inhibit MET or P13K (a signal transducer enzyme downstream from MET), which are being investigated to various degrees but not currently used clinically. For example, XL880 is a potent inhibitor of MET and VEGFR2. As used herein, the term “MET inhibitor” includes, but is not limited to: AMG 208, AMG 102, ARQ 197, AV-299, MetMab, GSK 1363089 (XL880), EMD 1214063, EMD 1204831, MGCD265, Crizotanib (PF-02341066), PF-04217903, MP470.

(3) COX2 inhibitors: As used herein, the term “COX2 inhibitor” includes, but is not limited to: selective COX2 inhibitors: celecoxib, rofecoxib, valdecoxib, lumiracoxib.

(4) Other non-steroidal anti-inflammatory drugs (NSAIDs), inhibiting both COX1 and COX2, such as ibuprofen, aspirin, indomethacin, and sulindac. Such drugs have also been shown to suppress NF-κB activation.

(5) Other NF-κB inhibitors. As used herein, the term “NF-κB inhibitor” includes, but is not limited to Arsenic trioxide (ATO), thalidomide and its analogues, resveratrol. In addition, it is thought that COX2 inhibitors also have an inhibitory effect on the NF-kB pathway. Therefore, NSAIDs, such as ibuprofen, aspirin, indomethacin, and sulindac were also shown to suppress NF-kB activation and as such are considered NF-kB inhibitors.

As used herein, the term “VEGF inhibitor” includes, but is not limited to: Bevacizumab, Cedaranib, Axitinib, Motesanib, BIBF 1120, Ramucirumab, VEGF Trap, Linifanib (ABT869), Tivozanib, BMS-690514, XL880, Sunitinib, Sorafenib, Brivanib, XL-184, Pazopanib.

As used herein, the term “targeted therapy” refers to a type of treatment that uses drugs or other substances, such as monoclonal antibodies or small-molecule inhibitors of specific enzymes, to identify and attack specific molecules, such as receptors. The examples of such are EGFR-TKIs (erlotinib, gefitinib), cetuximab, bevacizumab, etc.

As used herein, the terms “non-targeted chemotherapy” or “chemotherapy” refer to a therapy interfering with rapidly dividing cells either by interfering with DNA (such as alkylating agents, e.g. cisplatin, carboplatin, oxaliplatin or anti-metabolites, e.g. 5-fluoracil or pemetrexed, or topoisomerase inhibitors, such as irinotecan) or interfering with cell division (such as vinorelbine, docetaxel, paclitaxel).

As used herein, the term “prognostic” refers to a factor or a measurement that is associated with clinical outcome in the absence of therapy or with the application of standard therapy. It can be thought of as a measurement of a natural history of the disease.

The term “predictive” is a factor or a measurement which is associated with benefit or lack of benefit from a particular therapy. A predictive factor implies a differential benefit from the therapy that depends on the status of the predictive marker²

As used herein, the term “disease state” means a specific sub-type of the diagnosed condition that can be characterized by differential prognosis and/or differential response to therapy and/or specific molecular and/or metabolic characteristics.

Discussion

We have discovered that because the VeriStrat test is based on a signature obtained from the mass spectral data of a serum sample, it is able to measure general factors relating to cancer as opposed to most current biomarker-based tests. This fact allows new practical applications for the selection of treatment using the VeriStrat test, which are discussed below. In particular, the VeriStrat test results in a similar separation of survival curves between patient identified as VeriStrat “good” and patients identified as VeriStrat “poor” regardless of the mechanism of action of EGFR inhibition. In our previous work, the VeriStrat test used patient sample sets that were treated with the small molecule EGFR-tyrosine kinase inhibitors gefitinib (Iressa) and erlotinib (Tarceva), that inhibit the receptor by blocking the ATP-binding site of the enzyme¹. We observe similar separation between patients identified as VeriStrat “good” and patients identified as VeriStrat “poor” for another therapeutic agent targeting EGFR cetuximab (Erbitux) in both NSCLC and colorectal cancer (CRC)³. Cetuximab is an antibody which directly blocks the EGF receptor.

In addition, the VeriStrat test shows similar separation between patients identified as VeriStrat “good” and patients identified as VeriStrat “poor” across clinico-pathological characteristics. For example, the VeriStrat test can be used in patients whose tumor is an adenocarcinoma, as well as for patients whose tumor is a squamous cell carcinoma.

Also, the VeriStrat test shows separation between patients identified as VeriStrat “good” and patients identified as VeriStrat “poor” in a variety of solid epithelial tumors. We observed this in NSCLC, squamous cell cancer of the head and neck (SCCHN), and CRC³.

In addition, we found that the separation of survival curves by the VeriStrat test classification of in patients treated with non-targeted chemotherapy varies depending on details of the population, intervention type, and tumor type. There is evidence for separation in some non-targeted chemotherapy-treated sets, while the absence of separation in the others. There was also a strong separation seen in placebo arms, i.e., no intervention, indicating that the VeriStrat test has a prognostic component.

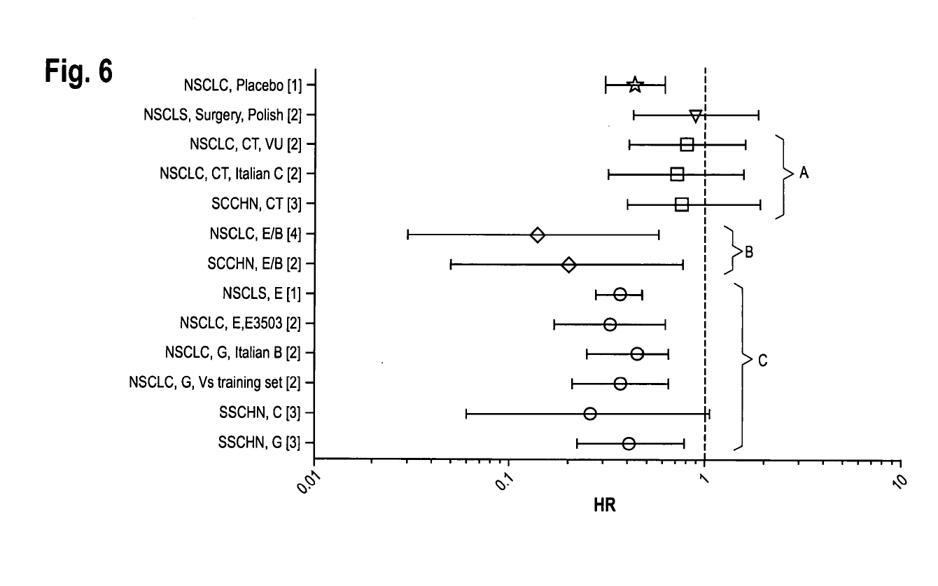

The forest plot of FIG. 6 summarizes data from all analysis of the VeriStrat test published or presented to date. It shows the hazard ratio (HR) for overall survival between VeriStrat “good” and VeriStrat “poor” patients for each treatment arm studied. The data can be seen to fall into groupings depending on treatment type. The range of hazard ratios obtained illustrates that VeriStrat is indeed indicative of better or worse outcome as a result of particular types of treatment, and hence has predictive power.

In FIG. 6, treatments are B=bevacizumab, C=cetuximab, CT=chemotherapy, E=erlotinib, G=gefitinib. Publications/presentations are [1] D. Carbone, 2nd European Lung Cancer Conference, April 2010, [2] data on file at Biodesix, updated from F. Taguchi et al., J Natl Cancer Inst. 2007 Jun. 6; 99(11):838-846′, [3] C. Chung et al., Cancer Epidemiol Biomarkers Prey. 2010 February; 19(2):358-65³, [4] D. Carbone et al., Lung Cancer 2010 September; 69(3):337-340⁴.

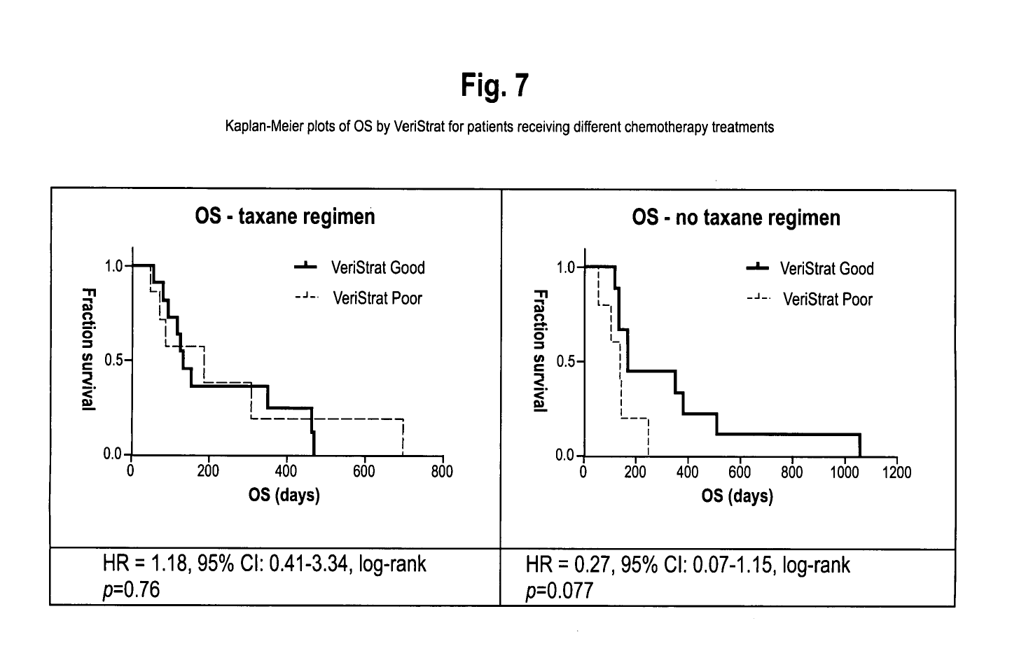

Re-analysis of the non-targeted chemotherapy treated population showed that while no apparent separation is observed in the subset of population treated with the taxanes, there is a separation between VeriStrat “good” and VeriStrat “poor” groups treated with the chemotherapy regimen containing no taxanes (see FIG. 7).

It is unusual that a test has such a large application range.

In summary, the following conclusions follow from the above discussion and FIGS. 6 and 7:
1. VeriStrat test shows a separation with a Hazard ratio between VeriStrat good and poor subgroups of around 0.45 for EGFR inhibitor (EGFR1) mono-therapies,
   independent of the mechanism of action of the EGFR1, e.g. for small molecule TKIs (erlotinib, gefitinib) and antibody (receptor) inhibitor based EGFRIs, e.g. cetuximab.
   independent of histological type, e.g. adeno carcinoma, and squamous cell carcinoma, and
   independent of organ, e.g. NSCLC, SCCHN, and CRC.
2. There is no observed significant correlation with other population characteristics:
   Not with genomic marker, e.g. EGFR mutation status or KRAS status.
   Not with population characteristics such as gender and race.
3. VeriStrat has a strong prognostic component exhibited by a separation between VeriStrat poor and VeriStrat good subgroups in the absence of treatment.
   However, there is no measurable treatment benefit of EGFRIs monotherapies in the VeriStrat “poor” subgroup, i.e. treatment with erlotinib is essentially equivalent to treatment with placebo in the VeriStrat poor subgroup, while there is a measurable treatment benefit of EGFRIs in the VeriStrat “good” subgroup.
   The effect of combination therapies depends on the particular drug combination and their effect on the interacting pathways.

All these facts taken together with the observation that only in the VeriStrat “poor” group specific peaks in the mass spectrum of the sample are observed, lead to the conclusion that VeriStrat defines a novel disease state of clinical significance (worse outcome) in solid epithelial tumors. The observed phenomena allow for some tentative conclusions on the molecular state of VeriStrat “poor” tumors: As EGFRIs are not effective in this class of patients, and as the effect is the same for both TKIs and antibody-based therapies, it is likely that in VeriStrat “poor” subjects, pathways below the receptors and the tyrosine-kinase domains are different from VeriStrat “good” subjects, i.e. upregulated. As we observe no correlation with KRAS mutation status, we further conclude that the affected pathway is below RAS.

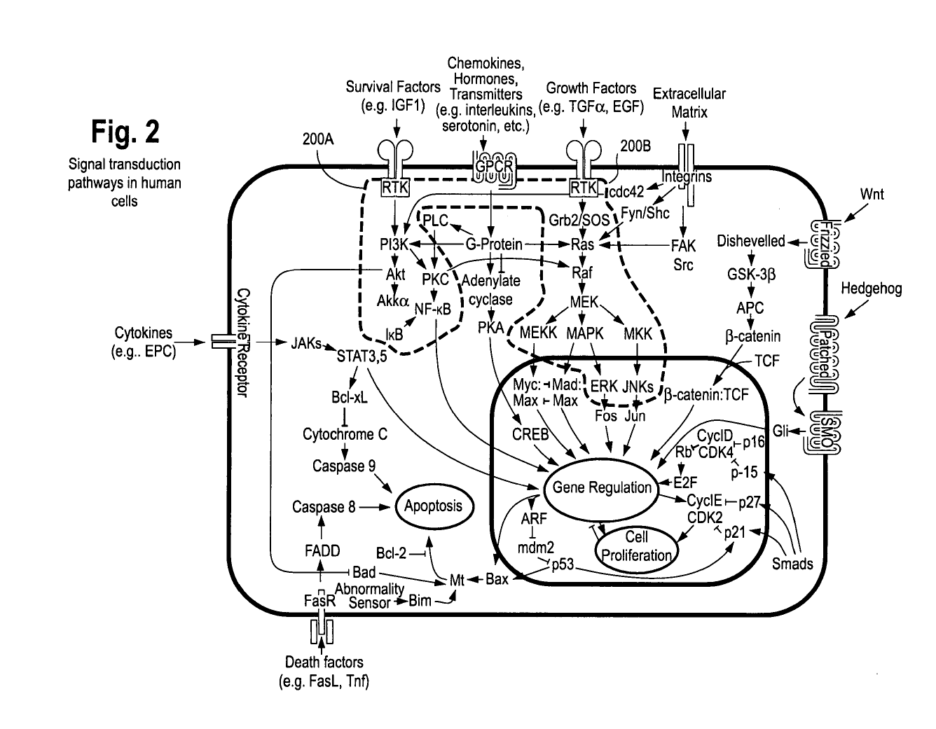

Based on the above observations, literature analysis and other lines of evidence, we present herein our understanding of which of the tumor cell's pathways are involved in the distinct characteristics of VeriStrat “poor” epithelial tumors. In brief, we propose that in patients identified as VeriStrat “poor” the VeriStrat test measures the activation of one or more pathways downstream from the receptors of EGF; likely candidate pathways include canonical and non-canonical MAPK, PI3K/Akt as well as reactions regulated by PKC (see FIG. 2 at 200A and 200B). The variability with respect to outcomes of chemotherapy and placebo controls indicates that the activation of these pathways by themselves could lead to worse prognosis, and may point to the involvement of the NF-κB transcription factor—an important regulator of cell survival, playing a key role in inflammatory processes and cancer progression and involved in the response to chemotherapy.

As a general matter, the VeriStrat test identifies a subset of population with worse prognosis (VeriStrat “poor”s) and will predict solid epithelial tumor cancer patient benefit from therapy with therapeutic agents or a combination of therapeutic agents targeting agonists of the receptors, receptors or proteins involved in MAPK pathways or the PKC (protein kinase C) upstream from or at Akt or ERK/JNK/p38 or PKC. EGFR inhibitors are the examples of such agents. Patients predicted to be likely to benefit from anti-EGFR agents are identified as VeriStrat “good” label; conversely patients predicted as not likely to benefit from anti-EGFR agents are identified with VeriStrat “poor” label. Patients having a VeriStrat “poor” label are not likely to obtain clinical benefit from therapy with such a therapeutic agent targeting at the receptors activating MAPK pathways; on the other hand, VeriStrat “poor” patients are likely to obtain clinical benefit from therapy or combination of therapies that prevents downstream, independent of the receptors, activation of these pathways.

The term MAPK (mitogen-activate protein kinase) here is used as name of at least three related cascades, not of a single enzyme (see FIG. 2).

As a corollary to the above statement, for patients that are associated with the VeriStrat “poor” label, the VeriStrat test is diagnostic for “poor” patients as a subgroup of cancer patients with a poor prognosis.

The consequences of the realizations can take the form of new methods, i.e., practical tests, for predicting whether cancer patients are likely or not likely to benefit from certain classes of drugs.

In one practical application, the invention can be considered as a method of identifying a solid epithelial tumor cancer patient as being likely to benefit from treatment with a therapeutic agent or a combination of therapeutic agents targeting agonists of the receptors, receptors or proteins involved in MAPK pathways or the PKC upstream from or at Akt or ERK/JNK/p38 or PKC or not likely to benefit from treatment with the therapeutic agent or the combination of therapeutic agents, comprising the steps of:

a) obtaining a mass spectrum from a blood-based sample from the solid epithelial tumor cancer patient;

b) performing one or more predefined pre-processing steps on the mass spectrum obtained in step a) (e.g., background subtraction, noise estimation, normalization and spectral alignment);

c) obtaining integrated intensity values of selected features in said spectrum at one or more predefined m/z ranges (and preferably the m/z ranges described below corresponding to the m/z peaks set forth in Table 1 below) after the pre-processing steps on the mass spectrum in step b) have been performed;

d) using the values obtained in step c) in classification algorithm (e.g., K-nearest neighbor) using a training set comprising class-labeled spectra produced from blood-based samples from other solid tumor patients to identify the patient as being either likely or not likely to benefit from treatment with the therapeutic agent or the combination of therapeutic agents.

As a specific example of overcoming of the resistance of VeriStrat “poor” patients to targeted therapy, the addition of COX2 inhibitors, e.g. celecoxib or rofecoxib, to EGFR-Is as a treatment regime may overcome the resistance of patients having a VeriStrat “poor” signature to EGFR-Is. The VeriStrat test may thus be used as an indicator to prescribe combination therapy including COX2 inhibitors and EGFR-Is.

As another specific example, the VeriStrat “poor” signature is believed to be associated with a specific activation of NF-κB, therefore the test can be used to select patients benefiting most from the NF-κB inhibitors, and, thus, to reduce unnecessary treatment and associated morbidities.

As another specific example, the VeriStrat “poor” signature is believed to be associated with little clinical benefit from specific non-targeted chemotherapies, specifically, the agents interfering with DNA replication and gene expression, such as cisplatin, gemcitabine or pemetrexed, possibly due to the involvement of NF-kB factor in this processes.

For patients classified as VeriStrat “poor”, addition of the agents, that (1) prevent downstream, independent from the receptors, activation of the MAPK pathways, such as COX2 inhibitors or (2) minimize the inflammatory host-responses, or addition of other targeted agents, that prevent cross-talk pathway activation, can overcome the resistance to the targeted agents.

The VeriStrat Test

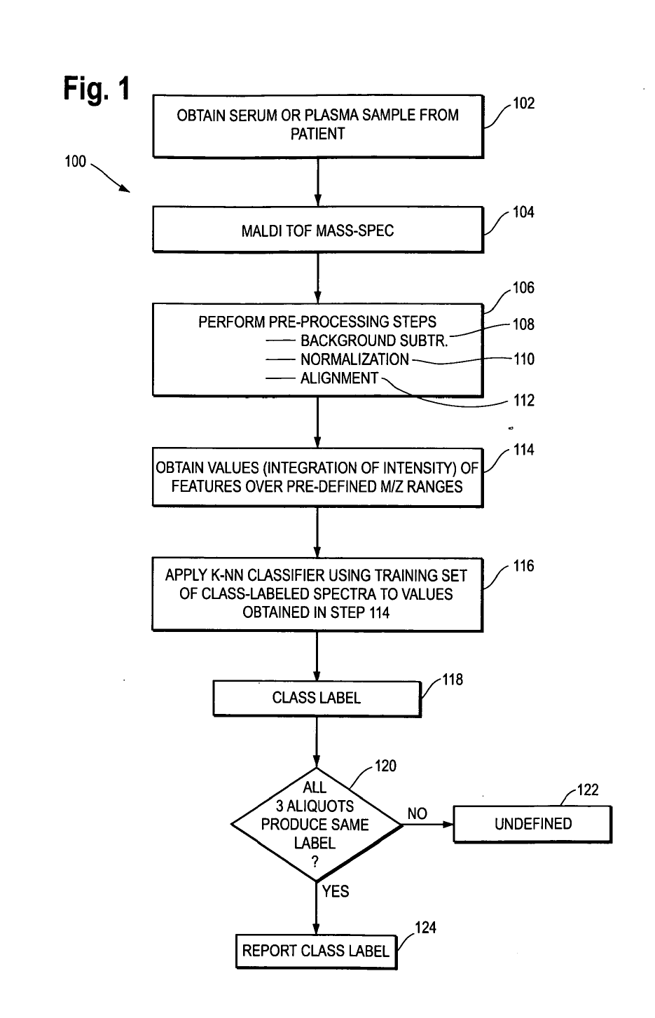

The methods for testing a blood-based sample of an solid epithelial tumor cancer patient in order to select such patient for treatment with certain therapeutic agent or a combination of therapeutic agents, such as agents targeting agonists of the receptors, receptors or proteins involved in MAPK pathways or the PKC pathway upstream from or at Akt or ERK/JNK/p38 or PKC in accordance with the present disclosure is illustrated in flow chart form in FIG. 1 as a process 100.

At step 102, a serum or plasma sample is obtained from the patient. In one embodiment, the serum samples are separated into three aliquots and the mass spectroscopy and subsequent steps 104, 106 (including sub-steps 108, 110 and 112), 114, 116 and 118 are performed independently on each of the aliquots. The number of aliquots can vary, for example there may be 4, 5 or 10 aliquots, and each aliquot is subject to the subsequent processing steps.

At step 104, the sample (aliquot) is subject to mass spectroscopy. A preferred method of mass spectroscopy is matrix assisted laser desorption ionization (MALDI) time of flight (TOF) mass spectroscopy, but other methods are possible. Mass spectroscopy produces data points that represent intensity values at a multitude of mass/charge (m/z) values, as is conventional in the art. In one example embodiment, the samples are thawed and centrifuged at 1500 rpm for five minutes at four degrees Celsius. Further, the serum samples may be diluted 1:10, or 1:5, in MilliQ water. Diluted samples may be spotted in randomly allocated positions on a MALDI plate in triplicate (i.e., on three different MALDI targets). After 0.75 ul of diluted serum is spotted on a MALDI plate, 0.75 ul of 35 mg/ml sinapinic acid (in 50% acetonitrile and 0.1% trifluoroacetic acid (TFA)) may be added and mixed by pipetting up and down five times. Plates may be allowed to dry at room temperature. It should be understood that other techniques and procedures may be utilized for preparing and processing serum in accordance with the principles of the present invention.

Mass spectra may be acquired for positive ions in linear mode using a Voyager DE-PRO or DE-STR MALDI TOF mass spectrometer with automated or manual collection of the spectra. Seventy five or one hundred spectra are collected from seven or five positions within each MALDI spot in order to generate an average of 525 or 500 spectra for each serum specimen. Spectra are externally calibrated using a mixture of protein standards (Insulin (bovine), thioredoxin (E. coli), and Apomyglobin (equine)).

At step 106, the spectra obtained in step 104 are subject to one or more pre-defined pre-processing steps. The pre-processing steps 106 are implemented in a general purpose computer using software instructions that operate on the mass spectral data obtained in step 104. The pre-processing steps 106 include background subtraction (step 108), normalization (step 110) and alignment (step 112). The step of background subtraction preferably involves generating a robust, asymmetrical estimate of background in the spectrum and subtracts the background from the spectrum. Step 108 uses the background subtraction techniques described in U.S. Pat. No. 7,736,905 B2 and U.S. patent application publication 2005/0267689, which are incorporated by reference herein. The normalization step 110 involves a normalization of the background subtracted spectrum. The normalization can take the form of a partial ion current normalization, or a total ion current normalization, as described in U.S. Pat. No. 7,736,905. Step 112 aligns the normalized, background subtracted spectrum to a predefined mass scale, as described in U.S. Pat. No. 7,736,905, which can be obtained from investigation of the training set used by the classifier.

Once the pre-processing steps 106 are performed, the process 100 proceeds to step 114 of obtaining values of selected features (peaks) in the spectrum over predefined m/z ranges. Using the peak-width settings of a peak finding algorithm, the normalized and background subtracted amplitudes may be integrated over these m/z ranges and assigned this integrated value (i.e., the area under the curve between the width of the feature) to a feature. For spectra where no peak has been detected within this m/z range, the integration range may be defined as the interval around the average m/z position of this feature with a width corresponding to the peak width at the current m/z position. This step is also disclosed in further detail in U.S. Pat. No. 7,736,905.

At step 114, as described in U.S. Pat. No. 7,736,905, the integrated values of features in the spectrum is obtained at one or more of the following m/z ranges:

5732 to 5795
5811 to 5875
6398 to 6469
11376 to 11515
11459 to 11599
11614 to 11756
11687 to 11831
11830 to 11976
12375 to 12529
23183 to 23525
23279 to 23622 and
65902 to 67502.

In a preferred embodiment, values are obtained at eight of these m/z ranges shown in Table 1 below, and optionally at all 12 of these ranges. The significance, and methods of discovery of these peaks, is explained in the U.S. Pat. No. 7,736,905.

At step 116, the values obtained at step 114 are supplied to a classifier, which in the illustrated embodiment is a K-nearest neighbor (KNN) classifier. The classifier makes use of a training set of class labeled spectra from a multitude of other patients (which may be NSCLC cancer patients, or other solid epithelial cancer patients, e.g., HNSCC, Breast Cancer). The application of the KNN classification algorithm to the values at 114 and the training set is explained in U.S. Pat. No. 7,736,905. Other classifiers can be used, including a probabilistic KNN classifier or other classifier.

At step 118, the classifier produces a label for the spectrum, either “good”, “poor” or “undefined”. As mentioned above, steps 104-118 are performed in parallel on the three separate aliquots from a given patient sample (or whatever number of aliquots are used). At step 120, a check is made to determine whether all the aliquots produce the same class label. If not, an undefined result is returned as indicated at step 122. If all aliquots produce the same label, the label is reported as indicated at step 124.

As described in this document, new and unexpected uses of the class label reported at step 124 are disclosed.

It will be understood that steps 106, 114, 116 and 118 are typically performed in a programmed general purpose computer using software coding the pre-processing step 106, the obtaining of spectral values in step 114, the application of the K-NN classification algorithm in step 116 and the generation of the class label in step 118. The training set of class labeled spectra used in step 116 is stored in memory in the computer or in a memory accessible to the computer.

The method and programmed computer may be advantageously implemented at a laboratory test processing center as described in our prior patent application publication U.S. Pat. No. 7,736,905.

The understanding of the mechanism of action of the VeriStrat test and its practical consequences stems from several sources, which will described further in this section.

Direct Evidence from Protein ID

VeriStrat measures the intensity of MALDI-TOF MS peaks from serum or plasma. In one embodiment, the VeriStrat signature consists of 8 mass spectral peaks described in Table 1, below. The classification is performed by estimating an intensity, i.e., a feature value, by integrating a sample's mass spectrum over pre-prescribed m/z ranges (see above listing and Table 1), and relating the observed set of 8 feature values to those from the training samples using a 7 nearest neighbor classification algorithm. This procedure uses the feature values in a non-linear combination, and does not allow for a definition of a one-dimensional score. Attempts to generate a score function from linear combinations of feature values have always been unsuccessful, and have always lead to worse performance. It appears that all or most of these eight features are useful in generating clinical utility.

It was thought that a determination of the peptide content of the used feature values might provide an understanding of mechanism of action of the VeriStrat test. However, this is complicated by the fact that the m/z resolution of the instrument is not sufficiently high to ensure that there is only one protein or peptide chain within a given m/z range. It also appears, that more than eight peptides constitute the eight peak signature, some of which are probably the post-translational modifications or oxidized forms of them same amino-acid sequences, while others may be still un-identified peptides. In addition, the feature values, i.e. the estimated peak intensity, do not simply correspond to the abundance of given analyte in the sample. This is due to the intricacies of the MALDI ionization process, where the number of ions hitting the detector is a function of both the abundance and the ionization probability of the analyte. This comparison of peaks (feature values) in a semi-quantitative manner, renders comparisons with standard methods for protein ID (LC-MS/MS) difficult.

TABLE 1
Peaks used in VeriStrat.

| Peak number | m/z |
|---|---|
| 1 | 5843 |
| 2 | 11445 |
| 3 | 11529 |
| 4 | 11685 |
| 5 | 11759 |
| 6 | 11903 |
| 7 | 12452 |
| 8 | 12579 |

Despite these difficulties, we have strong evidence that three of the peaks of Table 1 are related to serum amyloid A (SAA) isoforms. We have performed a differential gel (DIGE) analysis between pooled VeriStrat “good” and VeriStrat “poor” samples, and succeeded in isolating the peaks at m/z 11529 and 11685 with sufficient sequence coverage to identify them as SAA 19-122 and SAA 20-122. The theoretical masses agree well with the observed m/z values. The observed PI shift of 0.4 on the gel also agrees well with theoretical predictions. We also believe that the peak at m/z 5843 is the doubly charged form of the peak at 11685. These peaks have been observed by others⁵, (Ducet, et al. Electrophoresis 1996, 17, 866-876 Kiernan et al. FEBS Letters 2003, 537, 166-170). It is also possible that the peak at 11445 is another SAA isoforms related to a sequence of truncations from the C-terminus of the parent SAA protein.

While it is clear that other proteins or protein isoforms are present in the VeriStrat signature, it is possible that SAA isoforms play an important part in the mechanism of action of the VeriStrat test. In the following section, we provide a possible theory of the mechanism of action of the Veristrat test based on the discovery that SAA is a major part of at least three peaks in VeriStrat “poor” signature; known information on the interactions of SAA with certain receptors and of the biological consequences of these interactions, as well as the information on the presence of these receptors, functionally binding SAA, in various cancer cells. However, the present invention is not necessarily based on this theory, and such a theory is not meant to be limiting.

Prior art references on SAA as a biomarker in cancer: see references⁶⁻¹⁶.

SAA: Biological Functions and Involvement in Tumor Pathogenesis

Functions

A critical importance of the SAA family is suggested by the fact that SAA is a highly conserved sequence through evolution¹⁷, and the dramatic increase of SAA expression in response to infection, trauma or pathological processes. However, the exact biological functions of the SAA family are still not fully understood. SAA is involved in lipid transport and metabolism as a component of HDL, and probably plays a protective role in acute-phase of a disease¹⁸, while in chronic conditions SAA may become an adverse factor. Sustained high expression of SAA leads to amyloid A amyloidosis in some diseases, such as rheumatoid arthritis¹⁹. However, the range of clinically important function of SAA proteins is much broader, and includes implication in chronic inflammation and carcinogenesis. The latter two are closely related and are discussed in detail in the reviews of Vlasova and Moshkovskii²⁰ and Malle et al²¹.

Involvement of SAA in carcinogenesis can be attributed to its multifaceted biological activity: involvement in inflammation, including supporting chronic processes via pro-inflammatory gene expression activation and cytokine regulation, participation in extracellular matrix degradation, anti-apoptotic properties, and activation of specific pathways, including mitogen-activated protein kinase (MAPK), known to be intricately involved in carcinogenesis.

SAA is shown to be able to act as extracellular matrix (ECM) adhesion protein²² and to induce matrix metalloproteiniases (MMPs)¹⁸,²³, which play important role in ECM degradation and remodeling, and are associated with the tumorogenesis, metastases and tumor invasion.²⁴,²⁵.

Immune-related functions of SAA are defined by its cytokine-like activity. It can stimulate production of IL-8, TNF-α and IL-1β²⁶,²⁷ (which, probably, induces a positive feedback for the SAA expression), as well as IL-12 and 11-23, which play important role in cell-mediated immune response²⁸. It has also been shown that SAA can activate PI3K and p38 MAPK.

Involvement of SAA in regulation of inflammation can be associated with its ability to induce COX2 expression concurrently with activation of NF-κB and MAPK pathways.²⁹,³⁰. The principal interrelation of cancer and inflammation is a subject of numerous studies and reviews³¹⁻³⁷. The big body of recent data indicates that SAA may play an essential role as one of the mediators between the two processes, because of its ability to activate critical inflammatory and carcinogenic pathways, such as canonical and non-canonical MAPK pathways and of transcriptional factor NF-κB and, probably, participate in their cross-talk. The elevated levels of SAA, associated with VeriStrat signature, can be a used as a useful method of measuring activation of the pathways.

Receptors and Pathways, Associated with SAA Biological Activity

The NF-κB transcription factor is known to be constituitively activated in a large number of epithelial and hematologic malaignances and is considered to be essential for promoting inflammation-associated cancer³⁸,³⁹,⁴⁰, by regulating anti- and pro-apoptotic target genes, matrix-metalloprotease expression, angiogenesis and cell cycle⁴¹. On the other hand NF-κB can also exert pro-apoptotic genes activity and can cooperate with tumor suppressor p53 to induce apoptosis.⁴². The actual effect is dependent of the stimulus, cell-type, and the subunit involved⁴³. Anti- and pro-apoptotic effects of Rel/NF-κB factors are not necessarily alternative but can occur successively in the same cell, via the up-regulation of the same target gene⁴⁴. NF-κB is probably one of the main links between inflammation and cancer because of its association with induction of pro-inflammatory cytokines, such as IL-6 and TNF-α, and chemokines, including MMPs and COX-2³⁵,⁴⁵,⁴⁶. NF-κB activation can be induced by EGF: EGF stimulation prevents death receptor induced apoptosis trough NF-κB activation.

COX-2 over-expression is observed in broad range of pre-malignant, malignant and metastatic human epithelial cancers⁴⁷, including lung cancer⁴⁸. COX2 mediates, via prostaglandin E2 (PGE2), cell proliferation, angiogenesis, apoptosis, and cell migration, and also trans-activates tumorgenic signaling of mitogen-activated protein kinase MAPK cascade,⁴⁹,⁵⁰. COX2 trans-activates MAPK via Erk activation⁴⁹,⁹² The relationship is reciprocal: epidermal growth factor (EGF), acting trough MAPK pathway, dramatically inducts COX2 activity in some epithelial cells⁵¹. It was shown, that activation of EGFR by TGFα stimulates COX2 resulting in increased release of PGE2 and increased mitogenesis⁵².

The mitogen-activated protein kinase (MAPK) cascade plays a crucial role in normal cell biology, as well as in cancer development, because it transduces growth-stimulatory signals from activated growth factors receptors. The MAPK signal transduction is often initiated by binding of one of the growth factors to the membrane receptor tyrosine kinase receptor (RTK), leading to the engagement of Raf, MEK and extracellular-signal regulated kinase (ERK) kinases. Recent studies showed that signaling from RTK to ERK are much more complex than just a linear Ras-dependent pathway, and various signaling modulators have be identified that play a critical role in determining strength, duration and cell localization of ETK-mediated ERK signaling⁵⁰.

SAA functionally binds several receptors in various epithelial cells, and this binding can exert downstream activation of both NF-κB and MAPK pathways, that are described above and can lead to the resistance of VeriStrat “poor” patients to the specific treatments (as also discussed above). An overview of some of these receptors follows:

FPRL Receptors

FPRL receptors are expressed in various cells including hepatocytes⁵³ intestinal epithelium⁵⁴, and lung⁵⁵. SAA interacts with FPRL1—one of the classic G-protein coupled receptor—and triggers signaling networks, essential for regulation of cell function and epithelial proliferation and/or apoptosis. Binding of SAA to FPRL1, leads to activation and induction of interleukins. Involvement of FPRL activates protein kinase C (PKC) and the transcriptional factor NF-κB pathway³⁰, which is associated with inhibition of apoptosis and progression of cancer.⁵⁶,⁵⁷,⁴¹. It was also shown that binding of SAA to FPRL1 leads to apoptosis rescue of neutrophils and rheumatoid syniviocytes, which is mediated by phosphorylation of MAPK ERK 1/2, PI3K/Akt signaling, as well as STAT3 activation and release of intracellular Ca^{2+ 58},⁵⁹,⁶⁰, thereby promoting cell proliferation and survival.

SR-BI Receptors

The scavenger receptor B-I (SR-BI) was identified as a high density lipoprotein receptor, mediating selective cholesterol uptake.⁶¹ SR-BI is expressed most abundantly in steroidogenic tissues and liver, but also was upregulated in macropages and monocytes during inflammation; high SR-BI expression has been demonstrated in lipid-laden macrophages in human atherosclerotic lesion, also characterized by SAA presence. SAA was shown to promote cellular cholesterol efflux mediated by SR-BI⁶².

Baranova et al⁶³ demonstrated that specific binding of SAA (likely, in association with HDL) to SR-BI in HeLa and THP 1 (Human acute monocytic leukemia cell line) cells associated with phosphorylation of ERK1/2, and p38 MAPKs, and 11-8 secretions. Expression of SR-BI receptor was shown in different cells including human lung carcinoma cell lines⁶⁴.

RAGE

The Receptor for Advanced Glycation Endproducts (RAGE) is constantly expressed only in the lung at readily measurable levels but increases quickly at sites of inflammation, largely on inflammatory and epithelial cells. It is found that in epithelial cells RAGE, either as a membrane-bound or soluble protein, is markedly upregulated by stress. Perpetual signaling through RAGE induced survival pathways and diminished apoptosis, and (with ATP depletion) necrosis. This resulted in chronic inflammation which in many instances creates the setting in which epithelial malignancies arise.⁶⁵ RAGE overexpression was associated with prostate, colon and gastric tumors; while advanced stages of lung and esophageal cancer are characterized by downregulation of RAGE⁶⁶. In oral squamous cell carcinoma expression of RAGE was strongly associated with tumor progression and recurrence, and RAGE-positive patients showed significantly shorter disease-free survival. SAA, among other multiple ligands, was found to bind the receptor of advanced glycation end product (RAGE) and induce NF-κB through the ERK1/2 and p38 MAPK pathways (without induction of COX pathway)⁶⁷.

TLRs

Recent finding revealed that SAA could act as an endogenous agonist for toll-like receptors (TLRs) TLR4 and TLR2²¹. TLR4 was found to be expressed is some human cancer cells⁶⁸,⁶⁹. In lung cancer activation of TLR4 was shown to promote production of immunosuppressive cytokines TGF-beta, proangiogenic chemokine IL-8, and VEGF. Increased VEGF and IL-8 secretion is associated with p38MAPK activation.⁷⁰. Activation of TLR4 by SAA required phosphorylation of p42/44 and p38 MAPK⁷¹.

TLR2 was also shown to be a functional receptor for SAA. HeLa cells expressing TLR2 responded to SAA with potent activation of NF-κβ; SAA stimulation led to increased phosphorylation of ERK1/2 (P-ERK1/2), p38 MAPK (P-p38), and JNK (P-INK) MAPKs and accelerated IκBα (NFκB inhibitor) degradation in TLR2-HeLa cells⁷². Stimulation of NF-κB as result of a specific activation by SAA was demonstrated in macropahges.⁷³

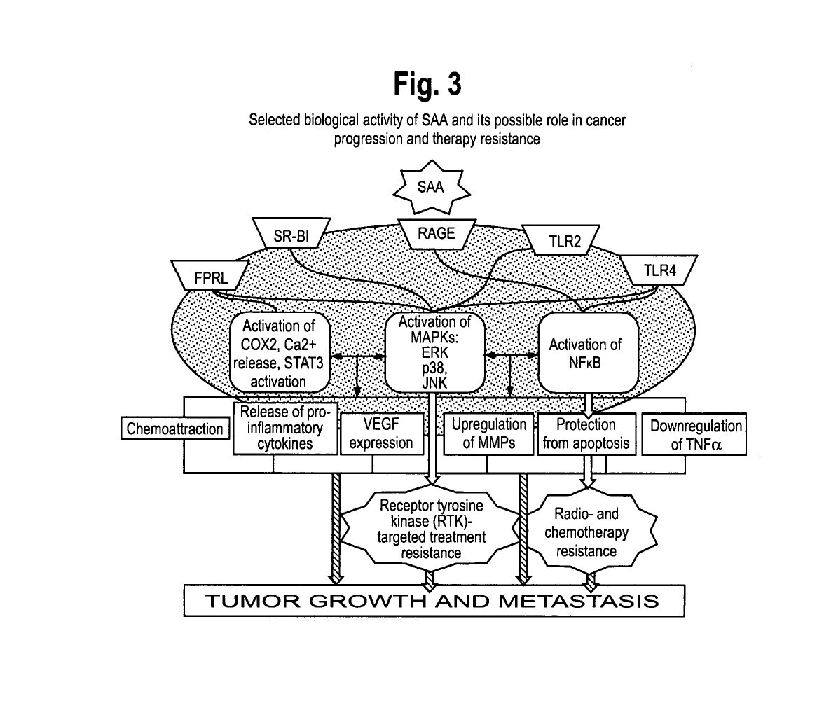

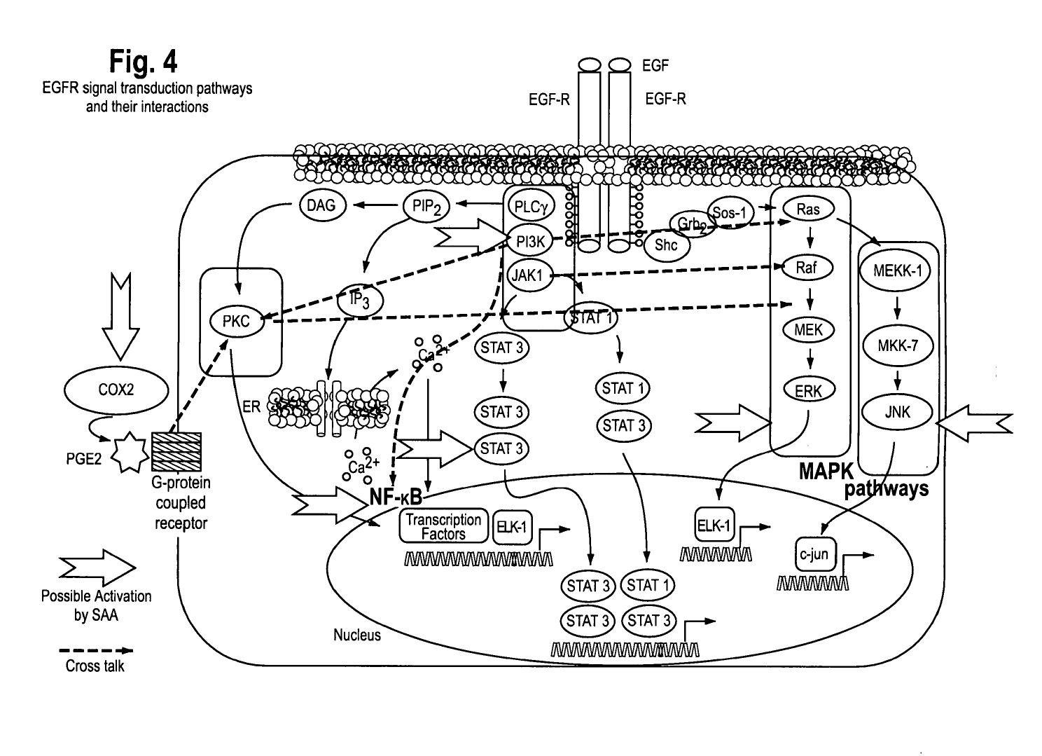

A simplified scheme of possible SAA interactions and its biological effects in cancer development and therapy resistance is presented in FIG. 3. As can be seen, the biological functions of SAA can be viewed in light of cross-talk of multiple pathways, triggered by interaction of SAA with various receptors, which eventually converge on activation of at least one of major MAPK pathways: ERK, p38 and JNK,²¹,⁴¹ and/or on NF-κB activation. Some of these interactions are illustrated on the schema of EGFR transduction pathway in FIG. 4.

EGFR is a tyrosine kinase receptor (TKR) activating several major downstream signaling pathways, including Ras-Raf-Mek and the pathway consisting of phosphoinositide 3-kinase (PI3K), Akt, and PKC. This in turn may have an effect on proliferation, survival, invasiveness, metastatic spread, and tumor angiogenesis interacting via multiple cross-talk connections with NF-κB transcription activation pathway and with the inflammatory pathways, e.g. induced by COX2. SAA may be able to activate these pathways independently of tyrosine-kinase receptor (shown by the wide arrows).

Overexpression and/or constitutive activation of EGFR is associated with numerous cancers, including brain, breast, intestinal and lung. Alteration of the components of the cascade lead to the activation of the pathways and are considered to be related to cancer induction and progression, e.g. activating mutations of EGFR kinase domain (in non-smokers) or of KRAS (in smokers) are associated with early development of lung cancer⁷⁴,⁷⁵. Ras protein is constitutively activated in about 25% of tumors, causing mitogeneic signaling independent of upstream regulation⁷⁶,⁷⁷. The large body of newly accumulated data suggest that non-linear signaling and trans-activation plays important role in cancer development and progression.

SAA Interactions and Resistance to Anti-Cancer Therapies

Chemotherapy, Radiation and Anti-Inflammatory Treatment

As discussed above and illustrated in FIGS. 3 and 4, interaction of SAA with a number of receptors leads to the activation of pathways associated with resistance to cancer therapies. The role of NF-κB in chemo- and radio-resistance has been discussed previously⁴¹. Inhibition of NF-κB conferred sensitivity to radiotherapy^{78, 79}, and death cytokines⁸⁰ by enhancing the apoptotic response. At the same time, exposure to radiation and certain chemotherapeutic drugs leads to NF-κB activation and subsequent resistance to apoptosis⁸¹,⁷⁹. Inhibition of chemotherapy (gemcitabine)-induced NF-κB activation was shown to restore sensitivity of NSCLC cell line to chemotherapy-induced apoptosis⁸²,⁸¹. On the other hand, in some cases, NF-κB was shown to be associated with sensitivity to chemotherapy, e.g. it has been suggested necessary for paclitaxel-induced cell death⁸².

Taking into account this information, one possible conclusion to draw from the increased SAA concentration in plasma or serum, characteristic for the VeriStrat “Poor” patients, is that the increased SAA may cause activation of NF-κB transcription factor and MAPK pathways. This may correlate with the cancers primary resistance to radiation therapy, and may affect the patient's response to chemotherapy. There are, however, a multitude of factors, which should be evaluated individually for each type of treatment and patient cohort.

NF-κB inhibitors, such as arsenic trioxide, curcumin, thalidomide were subject of numerous clinical trials. However, because NF-κB inhibitors also enhance the chemotherapy-induced apoptosis of normal hematopoietic progenitors, the use of NF-κB inhibitors as adjuvants in chemotherapy could delay bone marrow recovery. It should be considered that because NF-κB has a critical role in the activation of innate and adaptive immune responses, long-term use inhibitors is likely to be associated with a risk of immunodeficiency⁴¹.

If the VeriStrat “Poor” signature is, in fact, associated with a specific activation of NF-κB, this signature could be used to select patients benefiting most from the NF-κB inhibitors, and, may reduce unnecessary treatment and associated morbidities.

Receptor Tyrosine Kinases—Targeted Treatment

erbB Receptors and MAPK Pathways

EGFR and HER2 belong to the epidermal growth factor receptor (EGFR) family consisting of four members (EGFR (HER1), erbB4 (HER4), erbB3 (HER3), and erbB2 (HER2)). Since the majority of epithelial cancers exhibit abnormal activation of the epidermal growth factor receptor (EGFR) and HER2 receptor, specific inhibition of these receptors became a strategy of the targeted cancer therapy and are the subject of numerous studies.

In the absence of a ligand, EGFR receptors exist in a conformation that suppresses kinase activity. Ligand binding initiates a conformational alteration that unmasks a “dimerization loop”, triggering receptor dimerization. These transitions are relayed across the plasma membrane to activate kinase domains. Variations on this activation scheme are found in the ErbB family. ErbB-3 is not a functional kinase, but is able to transactivate dimer partners, whereas HER2/ErbB-2 is a ligand-less oncogenic receptor “locked” in the active conformation.

This dimerization results in the activation of tyrosine kinase function leading to the transduction of a signal through three major signaling pathways, and eventually to evasion of apoptosis, sustained angiogenesis, resistance to antigrowth signals, self-sufficiency in growth signals, and metastases.⁷⁷,⁸³.

Alteration of the components of the cascades leads to the activation of the pathways and is considered to be related to cancer induction and progression, e.g. activating mutations of EGFR kinase domain (in non-smokers) or of KRAS (in smokers) are associated with early development of lung cancer⁷⁴,⁷². Ras protein is constitutively activated in about 25% of tumors, causing mitogeneic signaling independent of upstream regulation⁷⁶,⁷⁷.

Several tyrosine kinase inhibitors are currently used in clinical practice for a variety of solid tumors, including two small molecule EGFR tyrosine kinase inhibitors—erlotinib and gefitinib, as well as the dual EGFR and HER2 inhibitor lapatinib. Also approved for clinical applications are the humanized monoclonal anti-HER2 antibody trastuzumab and two anti-EGFR antibodies—cetuximab and panitumumab.

The innate, as well as acquired resistance to tyrosine kinase inhibitors (small molecules, as well as monoclonal antibodies), reviewed in multiple publications, is attributed to various factors such as activating KRAS mutations, amplification of met-protoncogene⁸⁴, and T790M mutations. The diversity of cancer, and its ability to exhibit several pathways of resistance in response to targeted agents makes the prospect for curative therapy by a single agent more daunting⁸⁴, among other reasons, because of the possibility of activation of signaling independent of normal upstream interaction of ligands with their receptors. Growing evidence indicates the significance of coexpression of multiple tyrosine kinases, cross-talk of pathways downstream from the receptors, and downstream activation of the transduction cascades.

Trans-activation of the pathways was suggested as one of the mechanism of resistance in multiple studies. For example, insulin-like growth factor-I receptor (IGF-1R) signaling was shown to be able to compensate for EGFR blockade by gefitinib in human breast and prostate cancer cell lines⁸⁵. An alternative downstream signaling, in particular through Akt activation, such as by an oncogenic PIK3CA or by other RTK has been described as one of the mechanisms of resistance to TKIs in NSCLC.⁸⁶

Cappuzzo, et al⁸⁸ observed that sensitivity of patients with NSCLC to gefitinib was very low if the Akt was activated, while EGFR expression was negative, confirming that EGFR-independent activation may lead to gefitinib resistance.

We propose that the interactions of SAA, as measured by the VeriStrat test, can cause an RTK-independent activation of the MAPK cascade, and as a result, TKI resistance. This mechanism of the action of SAA may be direct or indirect. The direct action of SAA may be mediated by its binding to RAGE or TLR2 and TLR 4 receptors, leading to the activation of a classical MAPK pathway (by activation of JNK and p38). The presence of these receptors on the surfaces of various cancer cells, as well as in cancer associated cells and their interaction is reviewed in Malle, et al²¹. There is a direct evidence of activation of EGFR pathway as a result of activation of the TLR receptor⁶⁶.

Indirect action of SAA may be explained by acting via FPRL receptor, leading to the release of interleukins Il6, and Il8, which in turn, reacting with G-protein coupled receptor, activate PKC. (Activation of PKC leads to cell proliferation and vasopermeablity, and to activation of MEK in the MAPK pathway⁸⁶). Besides, it induces VEGF expression.

SAA is a ligand for TRL4 in lung endothelial cells and macrophages. Ligation of TLRs expressed in tumor cells reportedly also increases VEGF levels⁷⁰.

This information provides evidence for the presence of mechanisms responsible for downstream activation of all three major MAPK pathways by SAA. Downstream activation MAPK pathways is independent of RTKs and may lead to resistance to targeted inhibition upstream from the “crossing” checkpoint.

In view of the above, selection of patients most suitable for specific treatment, including combinational therapy, using the VeriStrat test, may be instrumental in overcoming some types of drug resistance.

Combinational Therapy and VeriStrat Signature

TKIs and COX2 Inhibitors

As was discussed above, SAA can induce expression of COX2. COX2 overexpression in lung cancer was first reported by Huang et al⁸⁷, it is observed in approximately 70% of adenocarcinomas⁸⁸, and was confirmed in many other studies.

A number of trials have demonstrated the cross-talk between COX2 and EGFR signaling pathways. As we discussed above, epidermal growth factor (EGF), acting through MAPK pathway, dramatically induces COX2 activity is some epithelial cells⁴⁷. Activation of EGFR by TGFα stimulates COX2 and leads to release of PGE2 and increased mitogenesis⁴⁸. On the other hand, prostaglandin E2 (PGE2), the product of COX2, can transactivate EGF receptor⁴⁵. In NSCLC PGE2 was demonstrated to activate MAPK/Erk pathway by intracellular cross-talk in EGFR-independent manner; the effect was mediated through G-protein coupled receptor and protein kinase C (PKC) and could contribute to EGFR-TKI resistance⁸⁹.

On the other hand COX2 inhibitors were shown to inhibit NF-κB pathway: celecoxib conferred its effect through suppression if Akt and IKK. In human non-small cell lung carcinoma, celecoxib was shown to suppress NF-κB, as well as TNF-induced JNK, p38 MAPK, and ERK activation through inhibition of IKK and Akt activation, leading to down-regulation of synthesis of COX-2 and other genes needed for inflammation, proliferation, and carcinogenesis⁴⁶,⁹⁰. Other NSAIDs, including aspirin and ibuprofen, were shown to act by suppressing IKK activation and IκBα degradation. Combined, these consideration provided strong rationale for addition of COX2 to standard cancer therapy.

The studies on combination of anti-inflammatory and tyrosine-kinase receptor-targeted therapy in NSCLC and its potential in overcoming EGFR-TKIs resistance has previously been reviewed^{90, 91}. The results of the trials were negative: response rate and survival of patients in combined therapy with gefitinib and celecoxib, and disease control rate in patients treated with rofecoxib and erlotinib⁹²,⁹³ were found similar to those observed in single agent treatment.

It is possible that the effect of the addition of COX inhibitors might be more pronounced in the VeriStrat “Poor” patients, due to the suggested up-regulating effect of SAA on this pathway. However, the magnitude of the effect is hard to predict because of the unknown magnitude of an effect of COX2 pathway inhibition on downstream MAPK activity and on NF-κB, and on their interplay. This hypothesis deserves further investigation.

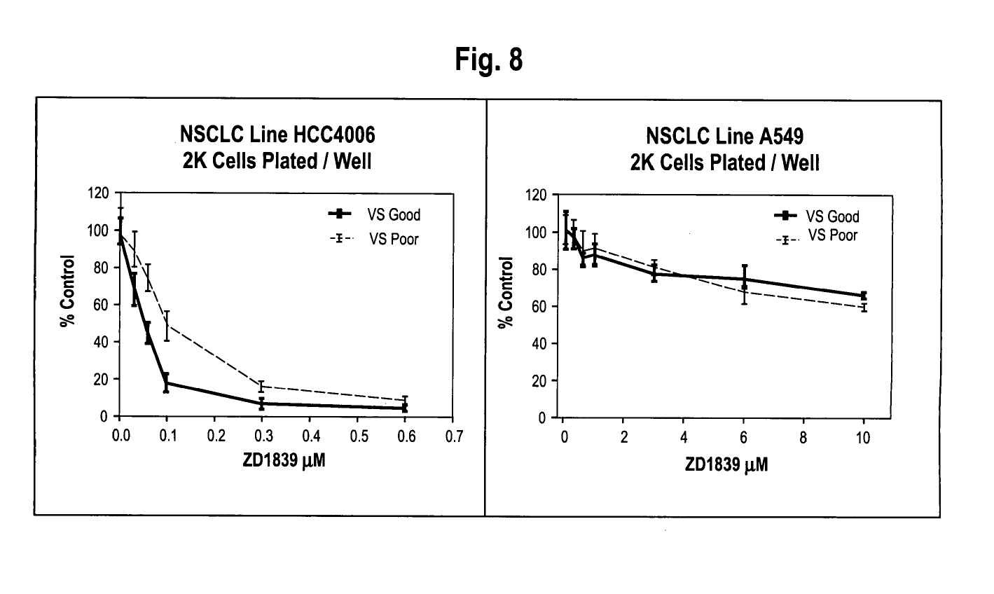

Cell Line Evidence (FIG. 8)

We have demonstrated that VeriStrat “poor” serum can cause a biological effect in tumor cells, in particular, it can increase resistance of cells to gefitinib in drug-sensitive cell lines. The experiments were carried out on the gefitinib sensitive line HCC4006 (it has EGFR exon 19 deletion) and the resistant line A549 (EGFR wild type). Human sera were from stage IIIB/IV NSCLC patients and characterized as VS ‘good” or “poor”. Pools were created by combining sera within each classification and used in growth inhibition assays. Cells were plated (10 replicates/drug concentration; 2,000 cells/well) using two media compositions; RPMI with 10% Good serum or RPMI with 10% Poor serum. After 24 hours, gefitinib was added and the plates were incubated for 6 days. The MTT assay was used to measure growth inhibition. The results are presented in Table 2 below and in FIG. 8.

TABLE 2

| | HCC4006* | | A549 | |
|---|---|---|---|---|
| | Good | Poor | Good | Poor |
| IC₅₀ μmol/L | 0.054 | 0.098 | >10 | >10 |
| % inhibition at 0.03 μmol/L | 32 | 10 | 0 | 0 |
| % inhibition at 0.06 μmol/L | 55 | 25 | 0 | 1 |
| % inhibition at 0.10 μmol/L | 82 | 52 | 3 | 0 |
| % inhibition at 0.30 μmol/L | 93 | 84 | 2 | 2 |
| % inhibition at 0.60 μmol/L | 96 | 93 | 14 | 11 |
| % inhibition at 1.0 μmol/L | ND | ND | 13 | 10 |
| % inhibition at 3.0 μmol/L | ND | ND | 22 | 20 |
| % inhibition at 6.0 μmol/L | ND | ND | 25 | 32 |
| % inhibition at 10.0 μmol/L | ND | ND | 34 | 40 |

*In HCC4006 P < 0.0001 for Good vs. Poor values by Mann-Whitney Test

FIG. 8 depicts graphs showing the growth of gefitinib sensitive cell line HCC4006, and gefitinib resistant cell line A549 in VeriStrat Poor and VeriStrat Good serum in presence of different concentrations of gefitinib. In FIG. 8, % Control was calculated from the ratio of the absorbance at the given concentration of gefitinib relative to the mean absorbance in the absence of the drug in the corresponding growth medium. Error bars correspond to standard deviation of the normalized measurements.

There was a relative decrease in inhibition of sensitive cells when grown in VeriStrat “poor” serum, but no significant change in resistant tumor cells. The results demonstrate that VeriStrat “poor” serum has a direct biological effect on tumor cells, and it is different from the effect of VeriStrat “good” serum. These results support our hypothesis of the VeriStrat mechanism, its relationship with the host-tumor interaction, and with the relative efficacy of targeted therapies in patient populations.

VeriStrat in Chemotherapies

As shown in FIG. 7, VeriStrat “poor” signature is associated with poor response to some non-targeted therapies, while not to others. VeriStrat classification is likely to be correlated with outcomes in chemotherapies, that interfere with DNA replication or with transcription of genes regulated by NF-kB (such as cisplatin, gemcitabine, etc), however concrete areas of VeriStrat usability in non-targeted therapies need to be determined experimentally.

An example of a practical application for the VeriStrat test then would be that it provides a method for predicting whether a cancer patient is not likely to benefit from administration of certain non-targeted chemotherapy regimes, such as one interacting with replication of DNA and/or activation of genes regulated by NF-kB transcription factor comprising: conducting the VeriStrat test on a sample (FIG. 1) and if the result is “poor” class label generating a result that the patient is not likely to benefit.

Taking into account the information from the literature that increased SAA is causing activation of NF-κB transcription factor, as well as the role of NF-kB activation in cancer progression and response to various therapies, VeriStrat signature may correlate with the cancer primary resistance to radiation therapy, and with patient's response to chemotherapy.

NF-κB inhibitors, such as arsenic trioxide, curcumin, thalidomide are being evaluated in clinical trials as anti-cancer agents. However, their usability can be limited by the absence of biomarkers of response to these agents, as well as by their side effects. VeriStrat can be useful as biomarker of the elevated activation of NF-kB, hence, for selection of patients (presumably, VeriStrat “poor”) potentially benefiting most from NF-kB inhibitors.

Summarizing all of the above, the present invention encompasses additional uses of the VeriStrat test of FIG. 1. As a general matter, the VeriStrat test will predict cancer patient benefit from therapy with any agent or combination of therapeutic agents, which is targeting agonists of the receptors, receptors or proteins involved in the MAPK pathways or the PKC (protein kinase C) pathway upstream from or at Akt or ERK/JNK/p38 or PKC. The magnitude of prediction will depend on a particular drug or drugs combination. The VeriStrat test will not predict effects of drugs targeting downstream regulations.

In one embodiment, the invention can be considered as a method of identifying a solid epithelial tumor cancer patient as being likely to benefit from treatment with a therapeutic agent or a combination of therapeutic agents targeting agonists of the receptors, receptors or proteins involved in MAPK pathways or the PKC pathway upstream from or at Akt or ERK/JNK/p38 or PKC or not likely to benefit from treatment with the therapeutic agent or the combination of therapeutic agents, comprising the steps of:

a) obtaining a mass spectrum from a blood-based sample from the solid epithelial tumor cancer patient;

b) performing one or more predefined pre-processing steps on the mass spectrum obtained in step a) (e.g., background subtraction, normalization and spectral alignment);

c) obtaining integrated intensity values of selected features in said spectrum at one or more predefined m/z ranges (and preferably the m/z ranges described previously corresponding to the m/z peaks set forth in Table 1) after the pre-processing steps on the mass spectrum in step b) have been performed;

d) using the values obtained in step c) in classification algorithm (e.g., K-nearest neighbor) using a training set comprising class-labeled spectra produced from blood-based samples from other solid epithelial tumor patients to identify the patient as being either likely or not likely to benefit from treatment with the therapeutic agent or the combination of therapeutic agents.

As a specific example the addition of targeted agents blocking the downstream activation of MAPK pathway to EGFR-Is may overcome the resistance of patients having a VeriStrat “poor” signature to EGFR-Is.

As another specific example, the addition of COX2 inhibitors, colecoxib or rofecoxib, to EGFR-Is as a treatment regime may overcome the resistance of patients having a VeriStrat “poor” signature to EGFR-Is. The VeriStrat test may thus be used as an indicator to prescribe combination therapy including COX2 inhibitors and EGFRIs. In a specific embodiment, the method for predicting whether a cancer patient is likely to benefit from administration of a COX2 inhibitor and a EGFR1 comprises the steps of a) obtaining a mass spectrum from a blood-based sample from the cancer patient; b) performing one or more predefined pre-processing steps on the mass spectrum obtained in step a) (e.g., background subtraction, normalization and spectral alignment); c) obtaining integrated intensity values of selected features in said spectrum at one or more predefined m/z ranges (and preferably the m/z ranges described previously corresponding to the m/z peaks set forth in Table 1) after the pre-processing steps on the mass spectrum in step b) have been performed; and d) using the values obtained in step c) in classification algorithm (e.g., K-nearest neighbor) using a training set comprising class-labeled spectra produced from blood-based samples from other solid epithelial tumor patients to identify the patient as being either likely or not likely to benefit from treatment by administration of a COX2 inhibitor and a EGFR-I. In particular, if the class label is “poor” the patient is indicated as likely to benefit.

As another specific example, the VeriStrat “Poor” signature is believed to be associated with a specific activation of NF-κB, therefore the test can be used to select patients benefiting most from the NF-κB inhibitors and the addition of COX2 inhibitors to the standard chemotherapy treatment, and, at the same time, to reduce unnecessary treatment and associated morbidities.

The methods of this disclosure can be implemented as a laboratory test center that receives blood-based samples from cancer patients (or mass spectral data from such samples), stores such mass spectral data in machine readable memory, and implements the processing and classification steps as shown in FIG. 1 in a machine, e.g., using a programmed computer, to generate the class label (VeriStrat “good” or “poor”), thereby providing the prediction of identification of the patient as likely to benefit from treatment from the therapeutic agent or combination of therapeutic agents as described above.

As another embodiment, the invention can be configured as an apparatus configured to identify or predict whether a cancer patient is likely to benefit from administration of the combination of a COX2 inhibitor and an EGFR inhibitor. The apparatus consists in combination of a storage device, computer memory or database, storing a mass spectrum of a blood-based sample from the cancer patient, and a processor (e.g., conventional CPU of a programmed general purpose computer) executing software instructions configured to a) perform one or more predefined pre-processing steps on the mass spectrum (See FIG. 1); b) obtain integrated intensity values of selected features in said spectrum at one or more predefined m/z ranges after the pre-processing steps on the mass spectrum in step a) have been performed (such as ranges encompassing the list of peaks of Table 1 or the m/z ranges set forth above); and c) use the values obtained in step b) in classification algorithm (e.g. KNN classification algorithm) using a training set comprising class-labeled spectra produced from blood-based samples from other cancer patients to identify the patient as being either likely or not likely to benefit from treatment by administration of a combination of a COX2 inhibitor and an EGFR inhibitor.

As another example, the invention can be embodied as an apparatus configured to identify a solid epithelial tumor cancer patient as being likely to benefit from treatment with a therapeutic agent or a combination of therapeutic agents targeting agonists of the receptors, receptors or proteins involved in MAPK (mitogen-activated protein kinase) pathways or the PKC (protein kinase C) pathway upstream from or at Akt or ERK/JNK/p38 or PKC or not likely to benefit from treatment with the therapeutic agent or combination of therapeutic agents. The apparatus takes the form of a storage device storing a mass spectrum of a blood-based sample from the solid epithelial tumor cancer patient, and a processor executing software instructions configured to a) perform one or more predefined pre-processing steps on the mass spectrum (See FIG. 1), b) obtain integrated intensity values of features in said mass spectrum at one or more predefined m/z ranges (such as ranges encompassing the list of peaks of Table 1 or the m/z ranges set forth above); and c) use the values obtained in step b) in a classification algorithm using a training set comprising class-labeled spectra produced from blood-based samples from other solid epithelial tumor cancer patients to identify the patient as being either likely or not likely to benefit from the therapeutic agent or a combination of therapeutic agents.

Further examples of the disclosed inventions are set forth in the appended claims.

### APPENDIX

### References Cited

1. Taguchi F, Solomon B, Gregorc V, et al. Mass spectrometry to classify non-small-cell lung cancer patients for clinical outcome after treatment with epidermal growth factor receptor tyrosine kinase inhibitors: a multicohort cross-institutional study. J Natl Cancer Inst 2007; 99:838-46.
2. Clark G M, Zborowski D M, Culbertson J L, et al. Clinical utility of epidermal growth factor receptor expression for selecting patients with advanced non-small cell lung cancer for treatment with erlotinib. J Thorac Oncol 2006; 1:837-46.
3. Chung C H, Seeley E H, Roder H, et al. Detection of tumor epidermal growth factor receptor pathway dependence by serum mass spectrometry in cancer patients. Cancer Epidemiol Biomarkers Prey 2010; 19:358-65.
4. Carbone D P, Salmon J S, Billheimer D, et al. VeriStrat((R)) classifier for survival and time to progression in non-small cell lung cancer (NSCLC) patients treated with erlotinib and bevacizumab. Lung Cancer 2009.
5. Kiernan U A, Tubbs K A, Nedelkov D, Niederkofler E E, Nelson R W. Detection of novel truncated forms of human serum amyloid A protein in human plasma. FEBS Lett 2003; 537:166-70.
6. Cremona M, Calabro E, Randi G, et al. Elevated levels of the acute-phase serum amyloid are associated with heightened lung cancer risk. Cancer 2010.
7. Benson M D, Eyanson S, Fineberg N S. Serum amyloid A in carcinoma of the lung. Cancer 1986; 57:1783-7.
8. Biran H, Friedman N, Neumann L, Pras M, Shainkin-Kestenbaum R. Serum amyloid A (SAA) variations in patients with cancer: correlation with disease activity, stage, primary site, and prognosis. J Clin Pathol 1986; 39:794-7.
9. Khan N, Cromer C J, Campa M, Patz E F, Jr. Clinical utility of serum amyloid A and macrophage migration inhibitory factor as serum biomarkers for the detection of nonsmall cell lung carcinoma. Cancer 2004; 101:379-84.
10. Cho W C, Yip T T, Yip C, et al. Identification of serum amyloid a protein as a potentially useful biomarker to monitor relapse of nasopharyngeal cancer by serum proteomic profiling. Clin Cancer Res 2004; 10:43-52.
11. Yokoi K, Shih L C, Kobayashi R, et al. Serum amyloid A as a tumor marker in sera of nude mice with orthotopic human pancreatic cancer and in plasma of patients with pancreatic cancer. Int J Oncol 2005; 27:1361-9.
12. Gutfeld O, Prus D, Ackerman Z, et al. Expression of serum amyloid A, in normal, dysplastic, and neoplastic human colonic mucosa: implication for a role in colonic tumorigenesis. J Histochem Cytochem 2006; 54:63-73.
13. Engwegen J Y, Mehra N, Haanen J B, et al. Validation of SELDI-TOF MS serum protein profiles for renal cell carcinoma in new populations. Lab Invest 2007; 87:161-72.
14. Dai S, Wang X, Liu L, et al. Discovery and identification of Serum Amyloid A protein elevated in lung cancer serum. Sci China C Life Sci 2007; 50:305-11.
15. Liu D H, Wang X M, Zhang L J, et al. Serum amyloid A protein: a potential biomarker correlated with clinical stage of lung cancer. Biomed Environ Sci 2007; 20:33-40.
16. Michaeli A, Finci-Yeheskel Z, Dishon S, Linke R P, Levin M, Urieli-Shoval S. Serum amyloid A enhances plasminogen activation: implication for a role in colon cancer. Biochem Biophys Res Commun 2008; 368:368-73.
17. Uhlar C M, Burgess C J, Sharp P M, Whitehead A S. Evolution of the serum amyloid A (SAA) protein superfamily. Genomics 1994; 19:228-35.
18. Uhlar C M, Whitehead A S. Serum amyloid A, the major vertebrate acute-phase reactant. Eur J Biochem 1999; 265:501-23.
19. Sipe J D. Amyloidosis. Annu Rev Biochem 1992; 61:947-75.
20. Vlasova M A, Moshkovskii S A. Molecular interactions of acute phase serum amyloid A: possible involvement in carcinogenesis. Biochemistry (Mosc) 2006; 71:1051-9.
21. Malle E, Sodin-Semrl S, Kovacevic A. Serum amyloid A: an acute-phase protein involved in tumour pathogenesis. Cell Mol Life Sci 2009; 66:9-26.
22. Preciado-Patt L, Levartowsky D, Prass M, Hershkoviz R, Lider O, Fridkin M. Inhibition of cell adhesion to glycoproteins of the extracellular matrix by peptides corresponding to serum amyloid A. Toward understanding the physiological role of an enigmatic protein. Eur J Biochem 1994; 223:35-42.
23. Migita K, Kawabe Y, Tominaga M, Origuchi T, Aoyagi T, Eguchi K. Serum amyloid A protein induces production of matrix metalloproteinases by human synovial fibroblasts. Lab Invest 1998; 78:535-9.
24. Hynes R O. The extracellular matrix: not just pretty fibrils. Science 2009; 326:1216-9.
25. Vihinen P, Ala-aho R, Kahari V M. Matrix metalloproteinases as therapeutic targets in cancer. Curr Cancer Drug Targets 2005; 5:203-20.
26. Furlaneto C J, Campa A. A novel function of serum amyloid A: a potent stimulus for the release of tumor necrosis factor-alpha, interleukin-1beta, and interleukin-8 by human blood neutrophil. Biochem Biophys Res Commun 2000; 268:405-8.
27. Patel H, Fellowes R, Coade S, Woo P. Human serum amyloid A has cytokine-like properties. Scand J Immunol 1998; 48:410-8.
28. He R, Shepard L W, Chen J, Pan Z K, Ye R D. Serum amyloid A is an endogenous ligand that differentially induces IL-12 and IL-23. J Immunol 2006; 177:4072-9.
29. Malle E, Bollmann A, Steinmetz A, Gemsa D, Leis H J, Sattler W. Serum amyloid A (SAA) protein enhances formation of cyclooxygenase metabolites of activated human monocytes. FEBS Lett 1997; 419:215-9.
30. Jijon H B, Madsen K L, Walker J W, Allard B, Jobin C. Serum amyloid A activates NF-kappaB and proinflammatory gene expression in human and murine intestinal epithelial cells. Eur J Immunol 2005; 35:718-26.
31. Coussens L M, Werb Z. Inflammation and cancer. Nature 2002; 420:860-7.
32. Farrow B, Sugiyama Y, Chen A, Uffort E, Nealon W, Mark Evers B. Inflammatory mechanisms contributing to pancreatic cancer development. Ann Surg 2004; 239:763-9; discussion 9-71.
33. Ditsworth D, Zong W X. NF-kappaB: key mediator of inflammation-associated cancer. Cancer Biol Ther 2004; 3:1214-6.
34. Balkwill F, Coussens L M. Cancer: an inflammatory link. Nature 2004; 431:405-6.
35. Lu H, Ouyang W, Huang C. Inflammation, a key event in cancer development. Mol Cancer Res 2006; 4:221-33.
36. Mantovani A, Allavena P, Sica A, Balkwill F. Cancer-related inflammation. Nature 2008; 454:436-44.
37. Lee J M, Yanagawa J, Peebles K A, Sharma S, Mao J T, Dubinett S M. Inflammation in lung carcinogenesis: new targets for lung cancer chemoprevention and treatment. Crit Rev Oncol Hematol 2008; 66:208-17.
38. Greten F R, Eckmann L, Greten T F, et al. IKKbeta links inflammation and tumorigenesis in a mouse model of colitis-associated cancer. Cell 2004; 118:285-96.
39. Pikarsky E, Porat R M, Stein I, et al. NF-kappaB functions as a tumour promoter in inflammation-associated cancer. Nature 2004; 431:461-6.
40. Karin M. The IkappaB kinase—a bridge between inflammation and cancer. Cell Res 2008; 18:334-42.
41. Lee C H, Jeon Y T, Kim S H, Song Y S. NF-kappaB as a potential molecular target for cancer therapy. Biofactors 2007; 29:19-35.
42. Graham B, Gibson S B. The two faces of NFkappaB in cell survival responses. Cell Cycle 2005; 4:1342-5.
43. Kaltschmidt B, Kaltschmidt C, Hofmann T G, Hehner S P, Droge W, Schmitz M L. The pro- or anti-apoptotic function of NF-kappaB is determined by the nature of the apoptotic stimulus. Eur J Biochem 2000; 267:3828-35.
44. Bernard D, Monte D, Vandenbunder B, Abbadie C. The c-R^{e1} transcription factor can both induce and inhibit apoptosis in the same cells via the upregulation of MnSOD. Oncogene 2002; 21:4392-402.
45. Li Q, Verma I M. NF-kappaB regulation in the immune system. Nat Rev Immunol 2002; 2:725-34.
46. Shishodia S, Koul D, Aggarwal B B. Cyclooxygenase (COX)-2 inhibitor celecoxib abrogates TNF-induced NF-kappa B activation through inhibition of activation of I kappa B alpha kinase and Akt in human non-small cell lung carcinoma: correlation with suppression of COX-2 synthesis. J Immunol 2004; 173:2011-22.
47. Koki A T, Khan N K, Woerner B M, et al. Characterization of cyclooxygenase-2 (COX-2) during tumorigenesis in human epithelial cancers: evidence for potential clinical utility of COX-2 inhibitors in epithelial cancers. Prostaglandins Leukot Essent Fatty Acids 2002; 66:13-8.
48. Soslow R A, Dannenberg A J, Rush D, et al. COX-2 is expressed in human pulmonary, colonic, and mammary tumors. Cancer 2000; 89:2637-45.
49. Pai R, Soreghan B, Szabo I L, Pavelka M, Baatar D, Tarnawski A S. Prostaglandin E2 transactivates EGF receptor: a novel mechanism for promoting colon cancer growth and gastrointestinal hypertrophy. Nat Med 2002; 8:289-93.
50. McKay M M, Morrison D K. Integrating signals from RTKs to ERK/MAPK. Oncogene 2007; 26:3113-21.
51. Richards J A, Petrel T A, Brueggemeier R W. Signaling pathways regulating aromatase and cyclooxygenases in normal and malignant breast cells. J Steroid Biochem Mol Biol 2002; 80:203-12.
52. Coffey R J, Hawkey C J, Damstrup L, et al. Epidermal growth factor receptor activation induces nuclear targeting of cyclooxygenase-2, basolateral release of prostaglandins, and mitogenesis in polarizing colon cancer cells. Proc Natl Acad Sci U S A 1997; 94:657-62.
53. Prossnitz E R, Ye R D. The N-formyl peptide receptor: a model for the study of chemoattractant receptor structure and function. Pharmacol Ther 1997; 74:73-102.
54. Babbin B A, Lee W Y, Parkos C A, et al. Annexin I regulates SKCO-15 cell invasion by signaling through formyl peptide receptors. J Biol Chem 2006; 281:19588-99.
55. Rescher U, Danielczyk A, Markoff A, Gerke V. Functional activation of the formyl peptide receptor by a new endogenous ligand in human lung A549 cells. J Immunol 2002; 169:1500-4.
56. Su S B, Gong W, Gao J L, et al. A seven-transmembrane, G protein-coupled receptor, FPRL1, mediates the chemotactic activity of serum amyloid A for human phagocytic cells. J Exp Med 1999; 189:395-402.
57. Biswas D K, Martin K J, McAlister C, et al. Apoptosis caused by chemotherapeutic inhibition of nuclear factor-kappaB activation. Cancer Res 2003; 63:290-5.
58. E1 Kebir D, Jozsef L, Khreiss T, et al. Aspirin-triggered lipoxins override the apoptosis-delaying action of serum amyloid A in human neutrophils: a novel mechanism for resolution of inflammation. J Immunol 2007; 179:616-22.
59. Lee H Y, Kim M K, Park K S, et al. Serum amyloid A induces contrary immune responses via formyl peptide receptor-like 1 in human monocytes. Mol Pharmacol 2006; 70:241-8.
60. Lee M S, Yoo S A, Cho C S, Suh P G, Kim W U, Ryu S H. Serum amyloid A binding to formyl peptide receptor-like 1 induces synovial hyperplasia and angiogenesis. J Immunol 2006; 177:5585-94.
61. Acton S, Rigotti A, Landschulz K T, Xu S, Hobbs H H, Krieger M. Identification of scavenger receptor SR-BI as a high density lipoprotein receptor. Science 1996; 271:518-20.
62. van der Westhuyzen D R, Cai L, de Beer M C, de Beer F C. Serum amyloid A promotes cholesterol efflux mediated by scavenger receptor B-I. J Biol Chem 2005; 280:35890-5.
63. Baranova 1N, Vishnyakova T G, Bocharov A V, et al. Serum amyloid A binding to CLA-1 (CD36 and LIMPII analogous-1) mediates serum amyloid A protein-induced activation of ERK1/2 and p38 mitogen-activated protein kinases. J Biol Chem 2005; 280:8031-40.
64. Hrzenjak A, Reicher H, Wintersperger A, et al Inhibition of lung carcinoma cell growth by high density lipoprotein-associated alpha-tocopheryl-succinate. Cell Mol Life Sci 2004; 61:1520-31.
65. Sparvero L J, Asafu-Adjei D, Kang R, et al. RAGE (Receptor for Advanced Glycation Endproducts), RAGE ligands, and their role in cancer and inflammation. J Transl Med 2009; 7:17.
66. Franklin W A. RAGE in lung tumors. Am J Respir Crit. Care Med 2007; 175:106-7.
67. Cai H, Song C, Endoh I, et al. Serum amyloid A induces monocyte tissue factor. J Immunol 2007; 178:1852-60.
68. Wang L, Liu Q, Sun Q, Zhang C, Chen T, Cao X. TLR4 signaling in cancer cells promotes chemoattraction of immature dendritic cells via autocrine CCL20. Biochem Biophys Res Commun 2008; 366:852-6.
69. Fukata M, Chen A, Vamadevan A S, et al. Toll-like receptor-4 promotes the development of colitis-associated colorectal tumors. Gastroenterology 2007; 133:1869-81.
70. He W, Liu Q, Wang L, Chen W, Li N, Cao X. TLR4 signaling promotes immune escape of human lung cancer cells by inducing immunosuppressive cytokines and apoptosis resistance. Mol Immunol 2007; 44:2850-9.
71. Sandri S, Rodriguez D, Gomes E, Monteiro H P, Russo M, Campa A. Is serum amyloid A an endogenous TLR4 agonist? J Leukoc Biol 2008; 83:1174-80.
72. Cheng N, He R, Tian J, Ye P P, Ye R D. Cutting edge: TLR2 is a functional receptor for acute-phase serum amyloid A. J Immunol 2008; 181:22-6.
73. He R L, Zhou J, Hanson C Z, Chen J, Cheng N, Ye R D. Serum amyloid A induces G-CSF expression and neutrophilia via Toll-like receptor 2. Blood 2009; 113:429-37.
74. Westra W H. Early glandular neoplasia of the lung. Respir Res 2000; 1:163-9.
75. Tang X, Shigematsu H, Bekele B N, et al. EGFR tyrosine kinase domain mutations are detected in histologically normal respiratory epithelium in lung cancer patients. Cancer Res 2005; 65:7568-72.
76. Medema R H, Bos J L. The role of p2 lras in receptor tyrosine kinase signaling. Crit. Rev Oncog 1993; 4:615-61.
77. Hanahan D, Weinberg R A. The hallmarks of cancer. Cell 2000; 100:57-70.
78. Shao R, Karunagaran D, Zhou B P, et al. Inhibition of nuclear factor-kappaB activity is involved in E1A-mediated sensitization of radiation-induced apoptosis. J Biol Chem 1997; 272:32739-42.
79. Yamagishi N, Miyakoshi J, Takebe H. Enhanced radiosensitivity by inhibition of nuclear factor kappa B activation in human malignant glioma cells. Int J Radiat Biol 1997; 72:157-62.
80. Luo J L, Kamata H, Karin M. The anti-death machinery in IKK/NF-kappaB signaling. J Clin Immunol 2005; 25:541-50.
81. Brach M A, Hass R, Sherman M L, Gunji H, Weichselbaum R, Kufe D. Ionizing radiation induces expression and binding activity of the nuclear factor kappa B. J Clin Invest 1991; 88:691-5.
82. Jones D R, Broad R M, Madrid L V, Baldwin A S, Jr., Mayo M W. Inhibition of NF-kappaB sensitizes non-small cell lung cancer cells to chemotherapy-induced apoptosis. Ann Thorac Surg 2000; 70:930-6; discussion 6-7.
83. Gazdar A F. Personalized Medicine and Inhibition of EGFR Signaling in Lung Cancer. N Engl J Med 2009.
84. Lynch T J, Jr., Blumenschein G R, Jr., Engelman J A, et al. Summary statement novel agents in the treatment of lung cancer: Fifth Cambridge Conference assessing opportunities for combination therapy. J Thorac Oncol 2008; 3:5107-12.
85. Jones H E, Goddard L, Gee J M, et al. Insulin-like growth factor-I receptor signalling and acquired resistance to gefitinib (ZD1839; Iressa) in human breast and prostate cancer cells. Endocr Relat Cancer 2004; 11:793-814.
86. Engelman J A, Janne P A. Mechanisms of acquired resistance to epidermal growth factor receptor tyrosine kinase inhibitors in non-small cell lung cancer. Clin Cancer Res 2008; 14:2895-9.
87. Huang M, Stolina M, Sharma S, et al. Non-small cell lung cancer cyclooxygenase-2-dependent regulation of cytokine balance in lymphocytes and macrophages: up-regulation of interleukin 10 and down-regulation of interleukin 12 production. Cancer Res 1998; 58:1208-16.
88. Hida T, Yatabe Y, Achiwa H, et al. Increased expression of cyclooxygenase 2 occurs frequently in human lung cancers, specifically in adenocarcinomas. Cancer Res 1998; 58:3761-4.
89. Krysan K, Reckamp K L, Dalwadi H, et al. Prostaglandin E2 activates mitogen-activated protein kinase/Erk pathway signaling and cell proliferation in non-small cell lung cancer cells in an epidermal growth factor receptor-independent manner. Cancer Res 2005; 65:6275-81.
90. Krysan K, Reckamp K L, Sharma S, Dubinett S M. The potential and rationale for COX-2 inhibitors in lung cancer. Anticancer Agents Med Chem 2006; 6:209-20.
91. Reckamp K L, Gardner B K, Figlin R A, et al. Tumor response to combination celecoxib and erlotinib therapy in non-small cell lung cancer is associated with a low baseline matrix metalloproteinase-9 and a decline in serum-soluble E-cadherin. J Thorac Oncol 2008; 3:117-24.
92. Gadgeel S M, Ruckdeschel J C, Heath E1, Heilbrun L K, Venkatramanamoorthy R, Wozniak A. Phase II study of gefitinib, an epidermal growth factor receptor tyrosine kinase inhibitor (EGFR-TKI), and celecoxib, a cyclooxygenase-2 (COX-2) inhibitor, in patients with platinum refractory non-small cell lung cancer (NSCLC). J Thorac Oncol 2007; 2:299-305.
93. O'Byrne K J, Danson S, Dunlop D, et al. Combination therapy with gefitinib and rofecoxib in patients with platinum-pretreated relapsed non small-cell lung cancer. J Clin Oncol 2007; 25:3266-73.

We claim:

1. A method of identifying a solid epithelial tumor cancer patient as being likely to benefit from treatment with a combination of therapeutic agents targeting agonists of the receptors, receptors or proteins involved in MAPK pathways or the PKC upstream from or at Akt or ERK/JNK/p38 or PKC or not likely to benefit from treatment with the combination of therapeutic agents, comprising the steps of:
a) obtaining a mass spectrum from a blood-based sample from the solid epithelial tumor cancer patient;
b) performing one or more predefined pre-processing steps on the mass spectrum obtained in step a);
c) obtaining integrated intensity values of selected features in said spectrum at one or more predefined m/z ranges after the pre-processing steps on the mass spectrum in step b) have been performed;
d) using the values obtained in step c) in classification algorithm using a training set comprising class-labeled spectra produced from blood-based samples from other solid tumor patients to identify the patient as being either likely or not likely to benefit from treatment with the combination of therapeutic agents;
wherein the combination of therapeutic agents comprises a tyrosine kinase inhibitor (TKI) and either a hepatocyte growth factor receptor (HGFR) inhibitor or a MET inhibitor.

2. The method of claim 1, wherein the HGRF inhibitor comprises a monoclonal antibody drug targeting HGF.

3. The method of claim 1, wherein the monoclonal antibody drug comprises AV-299.

4. The method of claim 3, wherein the TKI comprises an epidermal growth factor receptor tyrosine kinase inhibitor (EGFR-TKI).

5. The method of claim 4, wherein the EGFR-TKI is selected from the group of EGFR-TKIs consisting of erlotinib, gefitinib and cetuximab.

6. The method of claim 1, wherein the one or more predefined m/z ranges are selected from the group of m/z ranges consisting of: 5732 to 5795, 5811 to 5875, 6398 to 6469, 11376 to 11515, 11459 to 11599, 11614 to 11756, 11687 to 11831, 11830 to 11976, 12375 to 12529, 23183 to 23525, 23279 to 23622, and 65902 to 67502.
